(19) United States
(12) Patent Application Publication
(10) Pub. No.: US 2026/0263688 A1
(43) Pub. Date: Sep. 10, 2026

(54) Drive assembly for medicament delivery device

(71) Applicant: SHL Medical AG, Zug (CH)

(72) Inventors: Hsuan Wang, Zug (CH); Sebastian Karlsson, Nyköping (SE); Daniel Scott, Deerfield Beach, FL (US); Christoph Egloff, Zug (CH)

(21) Appl. No.: 18/871,918

(22) PCT Filed: May 26, 2023
(86) PCT No.: PCT/EP2023/064175

§ 371 Date: Dec. 5, 2024

Related U.S. Application Data
(60) Provisional application No. 63/350,468, filed on Jun. 9, 2022.

(30) Foreign Application Priority Data
Aug. 30, 2022 (EP) 22192878.1

(51) Int. Cl. A61M 5/142; A61M 5/168

(52) U.S. Cl. CPC A61M 5/14232; A61M 5/16877

(57) ABSTRACT

The present disclosure provides a drive assembly for a medicament delivery device. The drive assembly comprises a peristaltic pump assembly with a roller arranged inside a pump housing and configured to rotate around a first axis, A. The drive assembly comprises a flexible tube wherein at least a portion of the flexible tube is arranged transverse to the first axis, A, between the pump housing and the roller, and a delivery control mechanism configured to selectively switch the flexible tube between a delivery state and a non-delivery state by selectively engaging the flexible tube. In the delivery state, the rotation of the roller around the first axis, A, can move medicament contained within the medicament container and/or the flexible tube from the tube inlet to the tube outlet, by the rotation of the roller. In the non-delivery state, medicament contained within the medicament container and/or the flexible tube cannot be moved.

### CROSS-REFERENCE TO RELATED APPLICATIONS

The present application is a U.S. National Phase Application pursuant to 35 U.S.C. § 371 of International Application No. PCT/EP 2023/064175 filed May 26, 2023, which claims priority to (i) U.S. Provisional Application No. 63/350,468 filed Jun. 9, 2022, and (ii) EP Patent Application No. 22192878.1 filed Sep. 14, 2022. The entire disclosure contents of these applications are herewith incorporated by reference into the present application.

### TECHNICAL FIELD

The present invention generally relates to medical devices. In particular, it relates to a drive assembly for a medicament delivery device, and to a medicament delivery device comprising a drive assembly.

### BACKGROUND

Medicament delivery devices are used for delivering a large variety of different drugs for different purposes and in different volumes. Today, many drugs are being formulated for much larger volumes out of necessity or for competitive advantage. For these drugs, IV administration is currently the standard of care. In general, the IV treatment includes delivering of multiple medications that need to be administrated in a specific way when it comes to timing and dosage, e.g. different drugs may need to be taken in a specific sequence.

The IV injection is hard to be administrated at home by the patients themselves and therefore needs to be administrated by professional health care staff. Also, the existing subcutaneous injectors for large volumes needs a lot of setting up before patients can use them.

Hence, there is a need for medicament delivery devices which provides more versatility in terms of selection of drugs, dosage, and delivery speed, while being easier to use. For example, to provide mechanisms allowing certain doses of certain drugs to be administered in a specific sequence.

### SUMMARY

It is an object of the present invention to provide improved drive assemblies for medicament delivery devices, and to overcome or alleviate at least some of the drawbacks associated with the prior art.

In an aspect of the inventive concept, there is provided a drive assembly for a medicament delivery device. The drive assembly comprises a peristaltic pump assembly comprising a pump housing and a roller at least partially arranged inside the pump housing and configured to rotate around a first axis, A. The drive assembly further comprises a flexible tube extending between a tube inlet and a tube outlet, wherein the tube inlet is configured to be attached to a medicament container of the medicament delivery device, wherein at least a portion of the flexible tube is arranged transverse to the first axis, A, between the pump housing and the roller. The drive assembly further comprises a delivery control mechanism, configured to selectively switch the flexible tube between a delivery state and a non-delivery state by selectively engaging the flexible tube, wherein, in the delivery state, the rotation of the roller around the first axis, A, can move medicament contained within the medicament container and/or the flexible tube from the tube inlet to the tube outlet, by the rotation of the roller, and wherein, in the non-delivery state, medicament contained within the medicament container and/or the flexible tube cannot be moved from the tube inlet to the tube outlet by the rotation of the roller around the first axis, A.

Thus, the present invention is based on the common concept or idea of providing a drive assembly for a medicament delivery device comprising a peristaltic pump assembly, wherein a delivery control mechanism selectively engages the flexible tube(s) which is (are) being used by the peristaltic pump. The delivery control mechanism may switch the tube(s) between a delivery state and a non-delivery state, such that medicament present in the flexible tube(s) may be transported, or may not be transported, when the roller(s) of the peristaltic pump rotates. The delivery control mechanism may selectively engage the flexible tube(s) by applying pressure on the flexible tube(s) and at least partially squeeze the flexible tube(s), e.g. against a roller of the peristaltic pump. Alternatively and/or additionally, the delivery control mechanism may selectively engage the flexible tube(s) by sealing and/or un-sealing the flexible tube(s) to allow, and/or not allow, gas from inside the flexible tube(s) to leak from a lumen of the flexible tube(s), such that medicament present in the flexible tube(s) may be transported when the roller(s) of the peristaltic pump rotates, or not be transported when the roller(s) of the peristaltic pump rotates. It will be appreciated that the drive assembly may selectively engage one or more lumens of one or more flexible tubes. The selective engagement may switch one or more lumens of one or more flexible tubes between a delivery state and a non-delivery state.

It will be further appreciated that the drive assembly may allow medicament to be pumped from a chosen medicament container out of a plurality of medicament containers for a controllable time and at a controllable rate.

It will be further appreciated that the flexible tube is flexible, allowing it to change shape/form/condition/state when being engaged by the delivery control mechanism.

It will be further appreciated that the drive assembly may switch the peristaltic pump assembly between delivering medicament and not delivering medicament without changing the rotation of the roller of the peristaltic pump.

It will be further appreciated that the drive assembly provides easier administering of medicaments, allowing users that are not medical professionals to more easily administer the medicaments.

By selective engagement it may here be meant physically interacting, directly or indirectly. For example, the delivery control mechanism may influence, touch and/or connect with the flexible tube.

That the flexible tube is in a delivery state means that the flexible tube is in a state which allows medicament/fluids to be moved through the whole flexible tube from the tube inlet to the tube outlet, by a peristaltic pump/roller which acts upon at least a part of the flexible tube.

That the flexible tube is in a non-delivery state means that the flexible tube is in a state which does not allow medicament/fluids to be moved through the whole flexible tube from the tube inlet to the tube outlet, by a peristaltic pump/roller which acts upon at least a part of the flexible tube.

The drive assembly may be used for any medicament delivery device using a peristaltic pump, or be comprised in any medicament delivery device using a peristaltic pump. Furthermore, the medicament delivery device may comprise one or more containers for containing one or more different medicaments. Each container may have an inlet of a flexible tube connected to it, for transporting the medicament.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC 500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

According to an embodiment, the delivery control mechanism is configured to selectively switch the flexible tube between a plurality of delivery states and a non-delivery state by selectively engaging the flexible tube, wherein each of the plurality of delivery states may provide a different pump rate for the peristaltic pump assembly. In other words, the delivery control mechanism may have different levels of engagement with the flexible tube, and provide a different pump rate for the peristaltic pump based on the level of engagement. For example, the drive control mechanism may press the flexible tube more or less against the roller of the peristaltic pump, and switch the flexible tube to different delivery states, and thus provide different pump rates. The present embodiment is advantageous in that the pump rate of the medicament by the peristaltic pump assembly may be changed between a plurality of different delivery states without changing the setup or setting of the peristaltic pump assembly. For example, the roller may be rotating continuously at a constant speed while the delivery control mechanism may switch the delivery status between delivering and not delivering, while also being able to change the pump rate of the medicament.

According to an embodiment of the present invention, the delivery control mechanism comprises a lid element arranged inside the pump housing and configured to be movable relative the roller and the flexible tube in a direction transverse to the first axis, A, between a first lid position and second lid position, wherein in the first lid position a portion of the flexible tube is compressed between the roller and the lid, and wherein in the second lid position the lid is disengaged from the flexible tube. Such that the flexible tube is in the delivery state when the lid element is in the first lid position and the flexible tube is in the non-delivery state when the lid is in the second lid position. In other words, the delivery control mechanism may comprise a lid element that can be moved between at least two positions, wherein in one position the lid element presses against the flexible tube to compress it, and in another position the lid element does not press against the flexible tube such that the flexible tube is compressed. The lid element may be an object with many possible shapes and sizes. For example, the lid element may be an object with a shape according to the curvature of the flexible tube, which e.g. allows it to engage a larger surface of the flexible tube. The present embodiment is advantageous in that the flexible tube may be changed between a delivery state and non-delivery state in a convenient and versatile manner. This may be because the delivery of medicament may be stopped/started without changing the rotation of one or more rollers in the peristaltic pump assembly.

According to an embodiment of the present invention, the delivery control mechanism comprises a motor and/or a magnet unit, wherein the motor and the magnet unit are configured to move the lid element between the first and second lid position. The present embodiment is advantageous in that the flexible tube may be changed between states without the need for manual operation. Furthermore, the present embodiment is advantageous in that the drive assembly may be better automatized.

According to an embodiment of the present invention, the magnet unit is arranged in the pump housing, and wherein the lid element comprises a magnetic material, such that the magnet unit is arranged to provide a force on the lid element in a direction from the first lid position to the second lid position and/or in a direction from the second lid position to the first lid position. The force may be a magnetic force. The present embodiment is advantageous in that the magnet unit may provide a magnetic force acting upon the lid element, to push it and/or attract it towards the flexible tube. For example, the magnet unit may be placed on the opposite side of the roller relative to the lid element, thus allowing it to push or attract the lid element in a direction transverse to the first axis, A.

According to an embodiment of the present invention, the delivery control mechanism comprises a screw element configured to rotate around a fifth axis, E, parallel to first axis, A, the motor, wherein the motor is configured to rotate the screw element around the fifth axis, E, and a coupling part coupled to the screw element and the lid element, wherein the screw element is configured to move the coupling part when the screw element is rotated around the fifth axis, E, in a direction perpendicular to the fifth axis, and wherein the lid element is moved between the first and second lid position when the screw element rotates and the coupling part is moved. The present embodiment is advantageous in that it provides a convenient and versatile way of moving the lid element between the first and second position using a motor and/or magnet unit.

According to an embodiment of the present invention, the lid element comprises at least one protrusion, and the coupling part is coupled to the protrusion. The present embodiment is advantageous in that the forwarding/transferring of the force from the coupling part to the lid element may be more efficient and precise. Furthermore, the present embodiment is advantageous in that it the force may be concentrated, and e.g. be averted from more sensitive areas of the lid element.

According to an embodiment of the present invention, the screw element comprises a first set of threads and the coupling part comprises a first set of grooves, and wherein the first set of threads are configured to engage the first set of grooves, and wherein the screw element is configured to move the coupling part in a direction perpendicular to the fifth axis, E, by rotating around the fifth axis, E. The present embodiment is advantageous in that it provides an efficient way to move the coupling part relative the screw element.

According to an embodiment of the present invention, the delivery control mechanism comprises a motor and a magnet unit, and wherein the motor is configured to move the lid element in a first direction, and wherein the magnet unit is configured to provide a force on the lid element in the first direction or in a second direction opposite the first direction. In other words, the delivery control mechanism may comprise a motor configured to move the lid element between the first and second position in a first direction, and the magnet unit may be arranged to support and/or counteract the movement of the lid element performed by the motor. For example, the motor may move the lid element from the first lid position to the second lid position, and the magnet unit may move apply a force on the lid element to move it from the second lid position towards the first lid position.

According to an embodiment of the present invention, the peristaltic pump assembly comprises a shaft extending along the first axis, A, and configured to rotate around the first axis, A, and wherein the roller is arranged on the shaft and rotated around the first axis, A, when the shaft rotates. The present embodiment is advantageous in that a plurality of tubes may be switched between a delivery state and non-delivery state in an individual manner, while using the rotation of only one shaft.

According to an embodiment of the present invention, the drive assembly further comprises a motor, wherein the shaft is coupled to the motor, and the motor is configured to rotate the shaft around the first axis, A. The present embodiment is advantageous in that the rotation of the shaft and the rollers are more automized.

According to an embodiment of the present invention, the peristaltic pump assembly comprises at least two flexible tubes, and wherein the delivery control mechanism comprises at least two lid elements, and wherein each tube of the at least two flexible tubes can be compressed by a corresponding lid element of the at least two lid elements when the corresponding lid element is in the first lid position. The present embodiment is advantageous in that the drive assembly may control the flow of medicament for multiple flexible tubes in a convenient and versatile manner.

According to an embodiment of the present invention, at least a portion of a first flexible tube is arranged transverse to the first axis, A, between a first lid element and the roller, and at least a portion of a second flexible tube is arranged transverse to the first axis, A, between a second lid element and the roller. The first lid element is configured to be movable relative the roller and the first flexible tube in a direction transverse to the first axis, A, between the first lid position and the second lid position. The second lid element is configured to be movable relative the roller and second flexible tube in a direction transverse to the first axis, A, between the first lid position and the second lid position. The roller may comprise one or more rollers. For example, each lid element may have a corresponding lid element. Another example is that a plurality of lid elements has one and the same roller while another lid element has their own corresponding roller.

According to an embodiment of the present invention, the first and second lid element may be moved between the first and second lid position individually. The present embodiment is advantageous in that multiple flexible tubes may be switched between a delivery state and a non-delivery state individually. Furthermore, the present embodiment allows for delivery of different medicaments in series in a controlled and convenient manner. For example, firstly a first medicament is delivered via the first flexible tube connected to e.g. a first medicament container, and secondly a second medicament is delivered via the second flexible tube connected to e.g. a second medicament container.

According to an embodiment of the present invention, the drive assembly further comprises a control unit configured to control the movement of the lid element between the first and second lid position. The control unit may be and device/computer/tablet/interface which can receive instructions from a user and control the lid elements according to the instructions. Furthermore, the control unit may have pre-programmed/pre-defined programs on how to control the lid elements, both the position of the lid element and the timing of the movements of the lid element, which may provide a specific program/sequence for how to administer the drug(s) in the medicament delivery device to a patient. The control unit may receive signals from an interface, e.g. a button, or via a wire or wirelessly from an external device.

According to an embodiment of the present invention, the flexible tube comprises a breathable section arranged outside the pump housing and configured to be permeable to gas, and wherein the delivery control mechanism comprises a rotating element arranged circumferentially around the flexible tube comprising the breathable section, and configured to rotate relative the flexible tube, around a second axis, B. The rotating element comprises an inner surface facing the flexible tube, wherein the inner surface comprises a recess. The delivery control mechanism further comprises a sealing element arranged circumferentially around the second axis, B, between the inner surface and the flexible tube, wherein the sealing element completely covers the breathable section. The rotating element is configured to rotate between a first position and a second position, wherein in the first position the recess at least partially aligns with the breathable section, and wherein in the second position the breathable section is unaligned with the recess, such that the flexible tube is in the non-delivery state when the rotating element is in the first position and the flexible tube is in the delivery state when the rotating element is in the second position.

By completely covering the breathable section it is here meant to cover any segment/part/piece/portion of the breathable section without any play/gap, in order to create a sealing effect.

By align it is here meant overlapping and/or lined up/positioned next to. For example, if the breathable section is aligned with the recess, there is an overlap between the breathable section and the recess, such that gas may move between the breathable section and the recess. A further example is that the recess and the breathable section are aligned in parallel.

By unaligned it is here meant not overlapping and/or not lined up/positioned next to. For example, if the breathable section is unaligned with the recess, there is no overlap between the breathable section and the recess, and they are not aligned in parallel at all.

According to an embodiment of the present invention, the sealing element is attached to the inner surface. The present embodiment is advantageous in that it provides an improved gas-tight seal.

According to an embodiment of the present invention, the sealing element is flexible and is attached to the flexible tube. The present embodiment is advantageous in that it provides an improved gas-tight seal.

According to an embodiment of the present invention, the inner surface comprises an abutting portion arranged to abut the sealing element and/or the flexible tube and cover the breathable section when the rotating element is in the second position, such that a gas-tight seal is created with the sealing element for the breathable section. The present embodiment is advantageous in that no gas can be transported/leaked through the breathable section. The sealing element and the abutting portion may provide a gas-tight seal for the breathable section the rotating element is in the second position.

According to an embodiment of the present invention, the recess comprises a channel exiting the rotating element. The present embodiment is advantageous in that it improves gas transport in and out of the flexible tube via the breathable section and the channel to a space external to the flexible tube.

According to an embodiment of the present invention, the drive assembly comprises two flexible tubes, and wherein the flexible tubes are attached to one another, and wherein the flexible tubes extend between a respective tube inlet and a tube outlet, wherein each of the tube inlets is configured to be attached to a respective medicament container of the medicament delivery device. At least a portion of each of the flexible tubes is arranged transverse to the first axis, A, between the pump housing and the roller. A first of the two flexible tubes comprises a first breathable section and a second of the two flexible tubes comprises a second breathable section. The rotating element is arranged circumferentially around a segment of the two flexible tubes comprising the first and second breathable sections, and configured to rotate relative the two flexible tubes, around the second axis, B, wherein the recess of the inner surface is configured to be alignable with the first and second breathable section. The rotating element is configured to rotate between a third position and a fourth position, wherein in the third position, the recess at least partially aligns with the first breathable section and the second breathable section is unaligned with the recess, and wherein in the fourth position the recess at least partially aligns with the second breathable section and the first breathable section is unaligned with the recess, such that the first of the two flexible tubes is in the non-delivery state and the second of the two flexible tubes is in the delivery state when the rotating element is in the third position, and the second of the two flexible tubes is in the non-delivery state and the first of the two flexible tubes is in the delivery state when the rotating element is in the fourth position.

According to an embodiment of the present invention, the delivery control mechanism comprises a control element arranged to rotate around a third axis, C, parallel to the second axis, B. The control element comprises first cogs on a surface facing the rotating element, and wherein the rotating element comprises second cogs on a surface facing the first cogs, and wherein the first cogs are arranged to engage the second cogs when the control element is rotated around the third axis, C. The control element may be circular/annular in shape, e.g. it may be a control ring, wherein the rotating element is arranged inside the control ring. The control element may have any other shapes that can engage the rotating element, and wherein a movement of the control element engages the rotating element, such that the rotating element rotates.

According to an embodiment of the present invention, the control element is connected to a motor so that the control element may be rotated by the motor and/or manually rotated by an operator.

According to an embodiment of the present invention, the flexible tube comprises an outlet breathable section arranged outside the pump housing and configured to be permeable to gas, wherein an inlet segment of the flexible tube arranged on an inlet side of the peristaltic pump assembly comprises the breathable section and an outlet segment of the flexible tube arranged on an outlet side of the peristaltic pump assembly comprises the outlet breathable section. The delivery control mechanism further comprises a second rotating element arranged circumferentially around the outlet segment of flexible tube comprising the outlet breathable section, and configured to rotate relative the flexible tube, around a fourth axis, D. The rotating element comprises a second inner surface facing the flexible tube, wherein the inner surface comprises a second recess. The delivery control mechanism comprises a second sealing element arranged circumferentially around the fourth axis, D, between the inner surface and the flexible tube, wherein the second sealing element completely covers the outlet breathable section. The second rotating element is configured to rotate between a first position and a second position, wherein in the first position the second recess at least partially aligns with the outlet breathable section, and wherein in the second position the outlet breathable section is unaligned with the second recess, such that the flexible tube is in the non-delivery state when the rotating element is in the first position and the flexible tube is in the delivery state when the rotating element is in the second position. The second rotating element further comprises third cogs on a surface facing the first cogs, wherein the first cogs are arranged to engage the second cogs and the third cogs when the control element is rotated around the third axis, C, such that the flexible tube may switch to the delivery state or the non-delivery state by simultaneous selective engagement at the breathable section and at the outlet breathable section.

By “inlet side of the peristaltic pump assembly”, it is meant the part of the flexible tube where medicament travels from the tube inlet to the pump housing and the roller when the medicament is being delivered. By “outlet side of the peristaltic pump assembly”, it is meant the part of the flexible tube where medicament travels from the pump housing and the roller towards the tube outlet.

According to an embodiment of the present invention, the drive assembly further comprises a second control unit configured to control the rotation of the control element around the third axis, C. The second control unit may be and device/computer/tablet/interface which can receive instructions from a user and control the control element according to the instructions. Furthermore, the control unit may have pre-programmed/pre-defined programs on how to control the control element, both the position of the control element and the timing of the movements of the control element, which may provide a specific program/sequence for how to administer the drug(s) in the medicament delivery device to a patient. The control unit may receive signals from an interface, e.g. a button, or via a wire or wirelessly from an external device.

Further objectives of, features of, and advantages with the present invention will become apparent when studying the following detailed disclosure, the drawings, and the appended claims. Those skilled in the art realize that different features of the present invention, even if recited in different claims, can be combined in embodiments other than those described in the following.

### BRIEF DESCRIPTION OF THE DRAWINGS

This and other aspects of the present invention will now be described in more detail, with reference to the appended drawings showing embodiment(s) of the invention.

FIG. 1A-1B schematically show a cross-section of a drive assembly according to an exemplifying embodiment of the present invention,

FIG. 2 schematically shows a side-view drive assembly according to an exemplifying embodiment of the present invention,

FIG. 3 schematically shows a drive assembly according to an exemplifying embodiment of the present invention,

FIG. 4A-4B schematically shows a drive assembly according to an exemplifying embodiment of the present invention,

FIG. 5 schematically shows a drive assembly according to an exemplifying embodiment of the present invention,

FIG. 6A-6B schematically show a cross-section of a delivery control mechanism according to an exemplifying embodiment of the present invention,

FIG. 7 schematically shows a cross-section of a delivery control mechanism according to an exemplifying embodiment of the present invention,

FIG. 8 schematically shows a drive assembly according to according to an exemplifying embodiment of the present invention,

FIG. 9 schematically shows a cross-section of a delivery control mechanism according to an exemplifying embodiment of the present invention,

FIG. 10 schematically shows a cross-section of a delivery control mechanism and the flexible tube according to an exemplifying embodiment of the present invention

### DETAILED DESCRIPTION

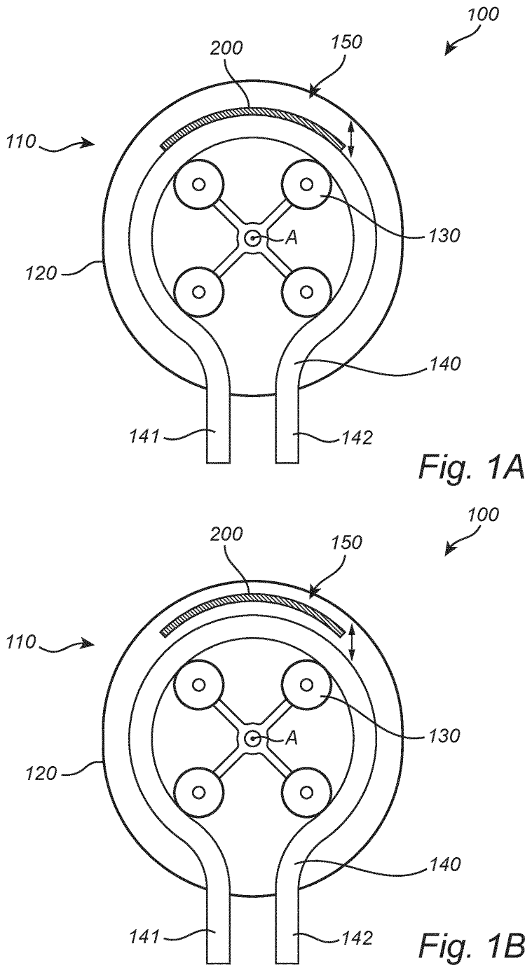

FIG. 1A-1B schematically show a cross-section of a drive assembly 100 according to according to an exemplifying embodiment of the present invention. The drive assembly 100 comprises a peristaltic pump assembly 110. The peristaltic pump assembly 110 comprises a pump housing 120. The peristaltic pump assembly 110 comprises four rollers 130 at least partially arranged inside the pump housing 120 and configured to rotate around a first axis, A. The rollers 130 may be attached to an axis/axle/shaft extending along the first axis, A. The rollers 130 may be attached to the axis/axle/shaft via an element, e.g. a shaft, protruding in a direction perpendicular to the first axis, A, such that the rollers 130 are arranged circumferentially around the first axis, A, distanced from each other in a radial direction of the first axis, A. It is to be understood that the peristaltic pump assembly 110 comprises one or more rollers 130. The drive assembly uses a flexible tube 140 extending between a tube inlet 141 and a tube outlet 142. The flexible tube 140 may be comprise a flexible plastic material, e.g. Polyvinyl chloride or Polyethylene. The tube inlet 141 is configured to be attached to a medicament container of the medicament delivery device, wherein at least a portion of the flexible tube 140 is arranged transverse to the first axis, A, between the pump housing 120 and the roller 130.

The drive assembly 100 further comprises a delivery control mechanism 150 configured to selectively switch the flexible tube 140 between a delivery state and a non-delivery state by selectively engaging the flexible tube 140. In the delivery state, the rotation of the roller 130 around the first axis, A, can move medicament contained within the medicament container and/or the flexible tube 140 from the tube inlet 141 to the tube outlet 142, by the rotation of the roller 130. In the non-delivery state, medicament contained within the medicament container and/or the flexible tube 140 cannot be moved from the tube inlet 141 to the tube outlet 142 by the rotation of the roller 130 around the first axis, A.

In FIGS. 1A and 1B, the drive assembly 100 further comprises a lid element 200 arranged inside the pump housing 120 and configured to be movable relative the roller 130, the flexible tube 140 and/or the pump housing 120 in a direction transverse to the first axis, A, between a first lid position and second lid position. In the first lid position a portion of the flexible tube 140 is compressed between the roller 130 and the lid element. In the second lid position the lid element 200 is disengaged from the flexible tube 140, such that the flexible tube 140 is in the delivery state when the lid element 200 is in the first lid position and the flexible tube 140 is in the non-delivery state when the lid element 200 is in the second lid position. By being disengaged, it may mean not touching and/or not applying pressure on the flexible tube 140, or at least not applying enough pressure to allow medicament to be moved in the flexible tube 140 by the peristaltic pump assembly 110.

The lid element 200 may be any object capable of engaging the flexible tube 140 and being sturdy enough to be pressed on to the flexible tube 140, allowing the roller 130 of the peristaltic pump assembly 110 to move medicament through the flexible tube. For example, the lid element 200 may be a lid, or lid object. The lid element 200 may comprise a surface following the curvature of the segment of the flexible tube 140 arranged between the pump housing 120 and the roller 130, i.e. the lid element 200 may comprise a surface extending in a radial direction of the first axis, A, e.g. in order to engage a larger part of the flexible tube 140 when being pressed down on to the flexible tube 140.

The delivery control mechanism 150 may be configured to selectively switch the flexible tube 140 between a plurality of delivery states and a non-delivery state by selectively engaging the flexible tube 140, wherein each of the plurality of delivery states may provide a different pump rate for the peristaltic pump assembly 110. For example, the lid element 200 may be moved to different first lid positions, such that it presses on to the flexible tube 140 with different pressures and compresses it to different degrees. Each different first lid position switches the flexible tube 140 to a different delivery state with a different pump rate for the peristaltic pump assembly 110. The lid element 200 may be moved between the first and second lid position by a manual mechanism and/or a motorized mechanism.

In FIG. 1A, the lid element 200 is in the first lid position and compresses the flexible tube 140 between the lid element 200 and the roller 130, i.e. the lid element 200 is engaging the flexible tube 140. Moving the lid element 200 to the first lid position switches the flexible tube 140 to a delivery state, allowing the peristaltic pump assembly 110 to transport medicament through the flexible tube 140.

In FIG. 1B, the lid element 200 is in the second lid position, i.e. the lid element 200 is disengaged from the flexible tube 140. Moving the lid element 200 to the second lid position switches the flexible tube 140 to a non-delivery state, preventing medicament from being transported through the flexible tube 140.

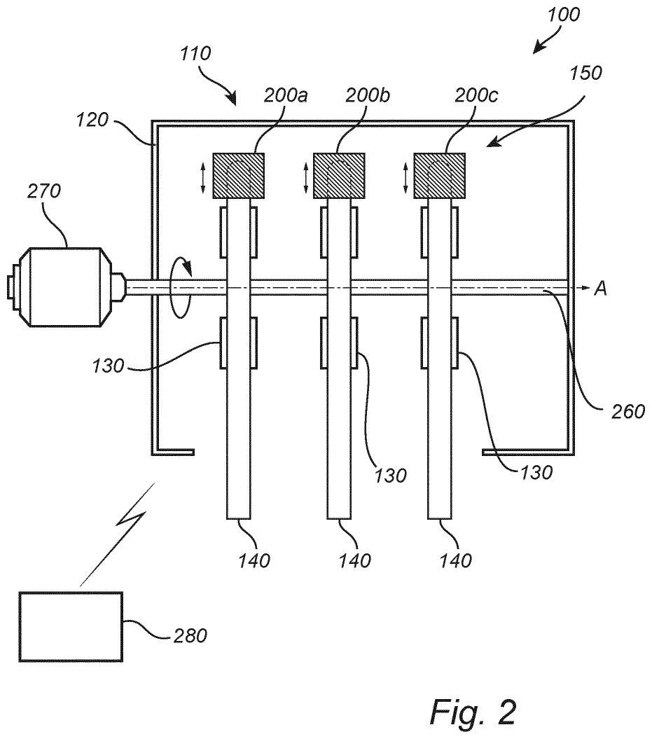

FIG. 2 schematically shows a side-view drive assembly 100 according to an exemplifying embodiment of the present invention. It should be noted that the drive assembly 100 shown in FIG. 2 has several features in common with the drive assembly 100 shown in FIG. 1A-B and it is hereby referred to FIG. 1 A-B and the associated text for an increased understanding of some of the features and/or functions of the drive assembly 100. The drive assembly 100 comprises a peristaltic pump assembly 110, comprising a pump housing 120 and a plurality of rollers 130 housed inside the pump housing 120. The drive assembly 100 further comprises a plurality of flexible tubes 140 and a delivery control mechanism 150. In FIG. 2, the plurality of flexible tubes 140 each have a corresponding lid element 200a, 200b, 200c configured to be movable relative the rollers 130 and the flexible tube 140 in a direction transverse to the first axis, A, between a first lid position and second lid position, in order to switch the corresponding flexible tube 140 between a non-delivery state and a delivery state by selectively engaging the flexible tube 140. The selective engagement here comprises compressing and de-compressing, i.e. not compressing, the flexible tube 140 between the lid element 200 and the roller 130.

Furthermore, in FIG. 2 the peristaltic pump assembly 110 comprises a shaft 260 extending along the first axis, A, and configured to rotate around the first axis, A, and wherein the plurality of rollers 130 are arranged on the shaft and rotates around the first axis, A, when the shaft 260 rotates. The plurality of rollers 130 may be attached/coupled/fixated to the shaft. Thus, all rollers 130 may be rotated by the same shaft 260. Furthermore, all rollers 130 may be rotating constantly and the medicament delivery may be controlled by individual movement of the lid elements 200a, 200b, 200c, between the first and second lid position.

In FIG. 2, the drive assembly 100 may comprise a first control unit 280 configured to control the movement of the lid elements 200a, 200b, 200c individually between the first and second lid position. Furthermore, the drive assembly 100 comprises a motor 270 configured to rotate the shaft 260. The first control unit 280 may control the motor 270 and the lid elements 200a, 200b, 200c.

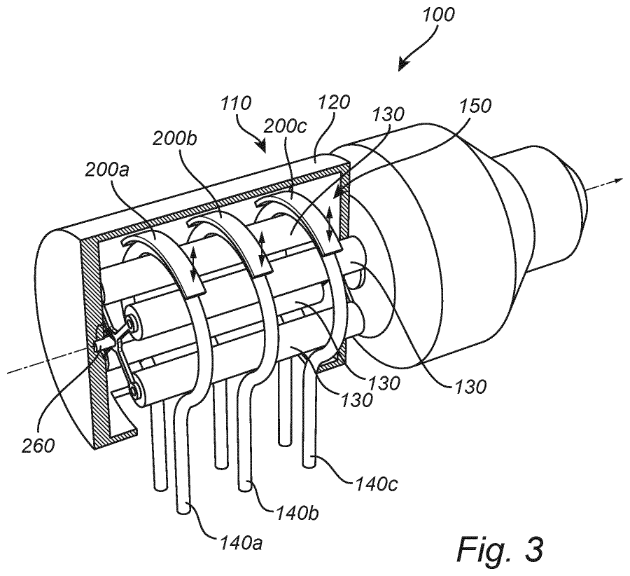

FIG. 3 schematically shows a drive assembly 100 according to an exemplifying embodiment of the present invention. It should be noted that the drive assembly 100 shown in FIG. 3 has several features in common with the drive assembly 100 shown in FIG. 1A-B and FIG. 2 and it is hereby referred to FIG. 1A-B and FIG. 2, and the associated texts for an increased understanding of some of the features and/or functions of the drive assembly 100. The drive assembly 100 comprises a peristaltic pump assembly 110, comprising a pump housing 120 and a plurality of rollers 130 housed inside the pump housing 120. The drive assembly 100 further comprises a plurality of flexible tubes 140 and a delivery control mechanism 150. In FIG. 2, the plurality of flexible tubes 140 each have a corresponding lid element 200a, 200b, 200c configured to switch the corresponding flexible tube 140a, 140b, 140c between a non-delivery state and a delivery state by selectively engaging the flexible tube 140, for example by compressing the flexible tube 140a, 140b, 140c between the lid element 200a, 200b, 200c and the roller 130. For example a first lid element 200a may compress a first flexible tube 140a, a second lid element 200b may compress a second flexible tube 140b and a third lid element 200c may compress a third flexible tube 140c, wherein the movement of the lid elements 200a, 200b, 200c may be individual.

In FIG. 3, the rollers 130 extend in a direction parallel with the first axis, A. The roller 130 may extend from a first lid position along the first axis A alongside a first lid element 200a to a second lid position along the first axis A alongside a third lid element 200c, such that a roller 130 is configured to move medicament through any number of the plurality of flexible tubes 140a, 140b, 140c when the flexible tube 140a, 140b, 140c is in a delivery state. The rollers 130 may be arranged on a shaft 260.

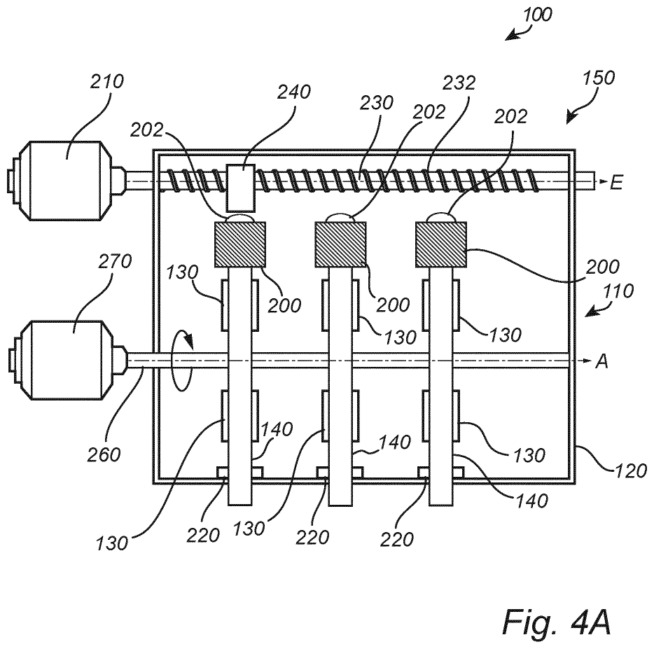

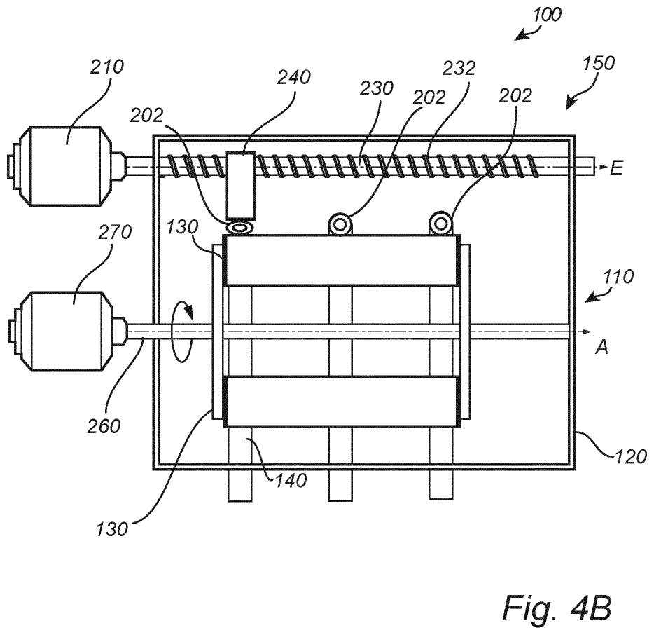

FIG. 4A-B schematically show a side-views of a drive assembly 100 according to an exemplifying embodiment of the present invention. It should be noted that the drive assembly 100 shown in FIG. 4 has several features in common with the drive assembly 100 shown in FIG. 1 A-B and FIG. 2-3, and it is hereby referred to FIG. 1 A-B and FIG. 2-3, and the associated texts for an increased understanding of some of the features and/or functions of the drive assembly 100

In FIG. 4A-B, the delivery control mechanism 150 comprises a screw element 230 configured to rotate around a fifth axis, E, parallel to the first axis, A. Furthermore, the delivery control mechanism 150 comprises a motor 210, wherein the motor is configured to rotate the screw element 230 around the fifth axis, E. The delivery control mechanism 150 further comprises a coupling part 240 coupled to the screw element 230 and the lid element 200. The coupling element 240 may be fixedly coupled to the lid element 200, to prevent rotation around of the coupling part. The screw element 230 is configured to move the coupling part 240 when the screw element 230 is rotated around the fifth axis, E, in a direction perpendicular to the fifth axis. The lid element 200 is moved between the first and second lid position when the screw element 230 rotates and the coupling part 240 is moved. It is to be understood that the delivery control mechanism 150 may comprise a plurality of screw elements 230, wherein each screw element 230 may enable the movement of the lid element 200 between the first and the second position. For example, each lid element 200 may be moved individually by an individual screw element 230.

The lid element 200 may comprise at least one protrusion 202, and the coupling part 240 may be coupled to the protrusion 202. The screw element 230 may comprise a first set of threads 232 and the coupling part 240 may comprises a first set of grooves (not shown). The first set of threads 232 are configured to engage the first set of grooves. It is to be understood that the first set of threads 232 may look different than what is illustrated in FIG. 4A-B. The first set of threads 232 and the first set of grooves may comprise any structure/configuration which allows them to engage and moves the coupling part 240 in a direction perpendicular to the fifth axis, E. The first set of grooves may be arranged on any surface of the coupling part 240. For example, the first set of grooves may be arranged on the outside of the coupling part 240 and/or in a cavity extending in the longitudinal direction of the coupling part 240. The screw element 230 is configured to move the coupling part 240 in a direction perpendicular to the fifth axis, E, by rotating around the fifth axis, E, and the first set of threads 232 are engaging the first set of grooves (not shown). In FIG. 4A, the motor 210 rotates a screw element 230 around the fifth axis, E, and the first set of threads 232 are configured to engage the first set of grooves 242 of the coupling part 240, such that the coupling part 240 may move back and forth towards the lid element 200 to engage, or disengage from, the lid element 200, or a protrusion 202 on the lid element 200, wherein the lid element 200 then engages the flexible tube 140 to compress and decompress the flexible tube 140. The protrusion 202 is here part of the lid element 200 and may comprise any shape.

Furthermore, in FIG. 4A, the delivery control mechanism 150 comprises magnet units 220 configured to move the lid element 200 between the first and second lid position. The lid element 200 may comprise a magnetic material, e.g. a metal, such that the magnet units 220 provide a force on the lid element 200 in a direction from the first lid position to the second lid position and/or in a direction from the second lid position to the first lid position. The delivery control mechanism 150 may comprise a motor 210 and a magnet unit 220, wherein the motor 220 is configured to move the lid element 200 in a first direction, and wherein the magnet unit 220 is configured to provide a force on the lid element 200 in the first direction or in a second direction opposite the first direction. The magnet unit 220 may be arranged inside the pump housing 120. The magnet unit 220 may be attached to the pump housing 120, to e.g. a base of the pump housing 120. The magnet unit 220 may be arranged on the opposite side of the roller 130 from the lid element 200. The magnet unit 220 may be configured to push the lid element 200 away from the flexible tube 140 and/or pull towards the flexible tube 140. The magnet unit 220 may comprise an electromagnet. The electromagnet may be controlled by a control unit. The control unit may switch the electromagnet on and off, in order to move the lid element 200.

In FIG. 4B, the lid element 200 comprises the coupling part 240. The motor 210 rotates a screw element 230 around the fifth axis, E, and the first set of threads 232 are configured to engage the first set of grooves 242 of the coupling part 240, such that the lid element 200 comprising the coupling part 240 may move back and forth towards the flexible tube 140, to engage, or disengage from, the flexible tube 140. In other words a roller 130 and the coupling part 240 are configured to squeeze the flexible tube 140 in between. Similarly, in this example, the lid element 200 is connected and configured to be driven by the delivery control mechanism 150 with the screw element 230 as described for the embodiment in FIG. 4A.

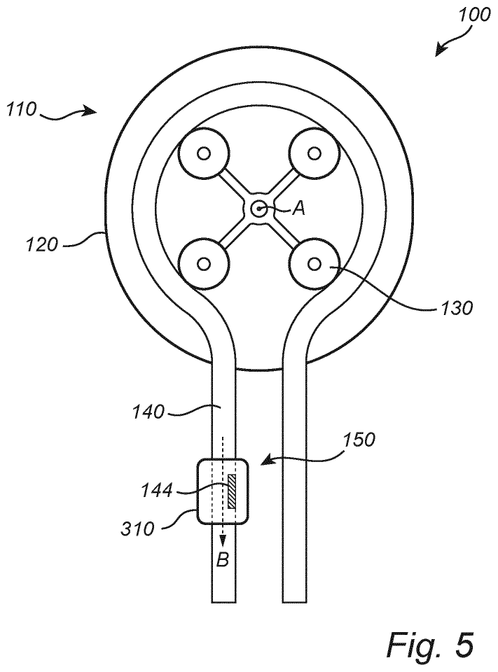

FIG. 5 schematically shows a drive assembly 100 according to an exemplifying embodiment of the present invention. The flexible tube 140 comprises a breathable section 144 arranged outside the pump housing 120 and configured to be permeable to gas. The delivery control mechanism 150 comprises a rotating element 310 arranged circumferentially around a segment of the flexible tube 140 comprising the breathable section 144, and configured to rotate relative the flexible tube 140, around a second axis, B.

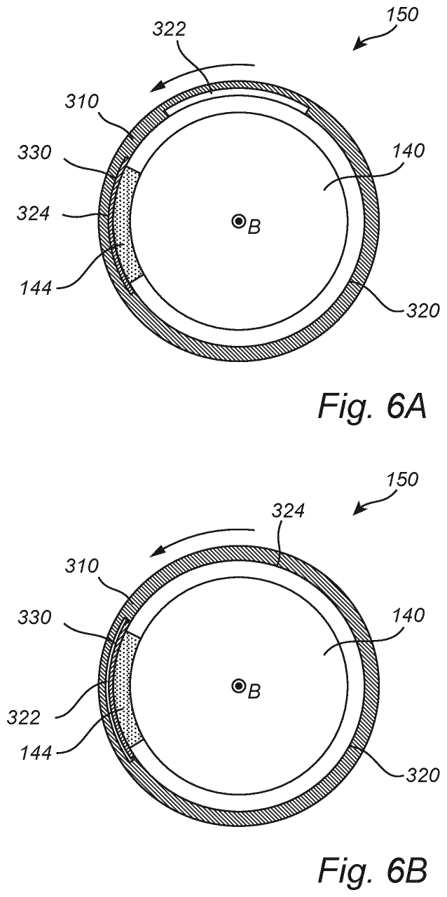

FIG. 6A-6B schematically show a cross-section of a delivery control mechanism 150 for example according to the embodiment in FIG. 5. The delivery control mechanism 150 comprises a rotating element 310. The rotating element 310 comprises an inner surface 320 facing the flexible tube 140, wherein the inner surface 320 comprises a recess 322. The recess 322 may comprise a channel exiting the rotating element 310. The recess 322 may be a conduit for gas moving to and/or from the flexible tube 140 via the breathable section 144. The recess 322 may comprise a cavity in the shape of a plane extending circumferentially around a part of the second axis, B.

The delivery control mechanism 150 comprises a sealing element 330 arranged circumferentially around the second axis, B, between the inner surface 320 and the flexible tube 140, wherein the sealing element 330 completely covers the breathable section 144. The sealing element 330 may abut a surface of the breathable section 144 facing the inner surface 320. The sealing element 330 may provide a sealing effect to keep gas from exiting/entering the flexible tube 140 via the breathable section 144.

The inner surface 320 may comprise an abutting portion 324 arranged to abut the sealing element 330 and/or the flexible tube 140 and cover the breathable section 144 when the rotating element 310 is in the second position. The abutting portion 324 may improve the sealing of gas inside the breathable section 144, such that no gas can enter or leak from the flexible tube 140 via the breathable section 144. The abutting portion 324 and the sealing element 330 may create a gas-tight seal. The abutting portion 324 may be the remaining part of the inner surface 320 when discarding the part with the recess 322. In other words, the inner surface 320 may comprise two portions, the abutting portion 324 and the recess 322, wherein the abutting portion 324 presses the sealing element 330 towards the flexible tube 140 and the recess 322 is distanced radially relative the second axis, B, from the sealing element 330 . The abutting portion 324 may be configured to compress the sealing element 330 between the abutting portion 324 and the flexible tube 140, such that a gas-tight seal is created with the sealing element 330 for the breathable section 144. The sealing element 330 may be attached to the inner surface 320 and cover the recess 322. The sealing element 330 may be flexible. The sealing element 330 may be attached to the flexible tube 140.

The recess 322 may be any cavity/void/hole/crack/slit that provides space for the sealing element 330 to expand/swell/stretch or move into, allowing gas to leave the flexible tube 140 via the breathable section 144.

The rotating element 310 is configured to rotate between a first position and a second position, wherein in the first position the recess 322 at least partially aligns with the breathable section 144, and wherein in the second position the breathable section 144 is unaligned with the recess 322, such that the flexible tube 140 is in the non-delivery state when the rotating element 310 is in the first position and the flexible tube 140 is in the delivery state when the rotating element 310 is in the second position. In other words, in the second position, the recess 322 at least partially aligns with the abutting portion 324 and does not align with the breathable section 144. Similarly, in the second position, the recess 322 may align with any part of the inner surface 320, except for the recess 322.

In FIG. 6A, the rotating element 310 is in the second position, i.e. is not aligned and/or overlapping with the breathable section 144 in any way. In FIG. 6A, no gas or liquid may leave the flexible tube 140 via the breathable section 144, because the sealing element 330 and the abutting portion 324 creates a gas-tight seal over the breathable section 144.

In FIG. 6B, the rotating element 310 is in the first position, i.e. at least partially aligned and overlapping with the breathable section 144. In FIG. 6B, the sealing element 330 may move into the recess 322 and gas may leave the flexible tube 140 via the breathable section 144. This may occur when the peristaltic pump assembly tries to pump medicament, and in this case the drive assembly cannot deliver any medicament.

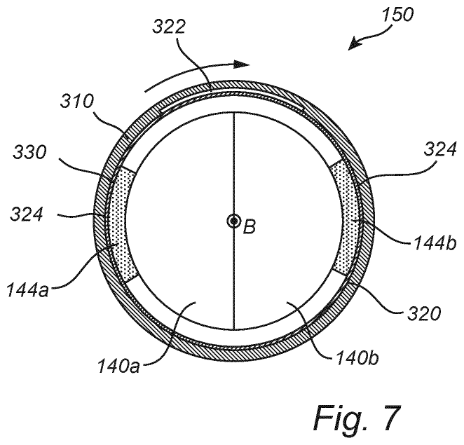

FIG. 7 schematically shows a cross-section of a delivery control mechanism 150 according to an exemplifying embodiment of the present invention. It should be noted that the delivery control mechanism 150 shown in FIG. 6A-B has several features in common with the delivery control mechanism 150 shown in FIG. 5 and 6A-B, and it is hereby referred to FIG. 5 and 6A-B, and the associated texts for an increased understanding of some of the features and/or functions of the delivery control mechanism 150.

In FIG. 7, the drive assembly comprises two flexible tubes 140a, 140b, wherein the flexible tubes 140a, 140b are attached to one another. The two flexible tubes 140a, 140b, may constitute one flexible tube with multiple lumen. The flexible tubes 140a, 140b may have a cross-section with the shape of a half-circle, and be combined to form one circular shape, as shown in FIG. 7. The flexible tubes 140a, 140b extend between a respective tube inlet and a tube outlet, wherein each of the tube inlets is configured to be attached to a respective medicament container of the medicament delivery device (not shown). At least a portion of each of the flexible tubes 140a, 140b is arranged transverse to the first axis, A, between the pump housing and the roller (not shown).

A first of the two flexible tubes comprises a first breathable section 144a and a second of the two flexible tubes 140a, 140b comprises a second breathable section 144b. The rotating element 310 is arranged circumferentially around a segment of the two flexible tubes 140a, 140b comprising the first and second breathable sections 144a,144b. The rotating element 310 is configured to rotate relative the two flexible tubes 140a, 140b, around the second axis, B. The recess 322 of the inner surface 320 is configured to be alignable with the first and second breathable section 144a, 144b.

The rotating element 310 is configured to rotate between a third position and a fourth position. In the third position, the recess 322 at least partially aligns with the first breathable section 144a and the second breathable section 144b is unaligned with the recess 322. In the fourth position the recess 322 at least partially aligns with the second breathable section 144b and the first breathable section 144a is unaligned with the recess 322. In FIG. 7, the inner surface 320 comprises an abutting portion 324. The rotating element 310 may be configured to rotate between the third, the fourth and a fifth position. In the fifth position the recess 322 does not align with the first or the second breathable section 144a, 144b, such that both the flexible tubes 140a, 140b are in a delivery state. Furthermore, the abutting portion 324, in the third position, abuts the sealing element 330 and the flexible tube 140b and covers the breathable section 144b, such that a gas-tight seal is created with the sealing element 330 and the abutting portion 324 for the breathable section 144b. In the third position the flexible tube 140a is in the non-delivery state, and the flexible tube 140b is in the delivery state. Furthermore, the abutting portion 324, in the fourth position, abuts the sealing element 330 and the flexible tube 140a and covers the breathable section 144a, such that a gas-tight seal is created with the sealing element 330 and the abutting portion 324 for the breathable section 144a. In the fourth position the flexible tube 140b is in the non-delivery state, and the flexible tube 140a is in the delivery state. In the fifth position, the abutting portion 324 abuts the sealing element 330, the flexible tube 140a and the flexible tube 140b, and covers both the breathable sections 144a and 144b, such that the flexible tubes 140a and 140b are both in a delivery state.

The third and fourth position are chosen, such that the first flexible tube 140a is in the non-delivery state and the second flexible tube 140b is in the delivery state when the rotating element 310 is in the third position, and the second flexible tube 140b is in the non-delivery state and the first flexible tube 140a is in the delivery state when the rotating element 310 is in the fourth position.

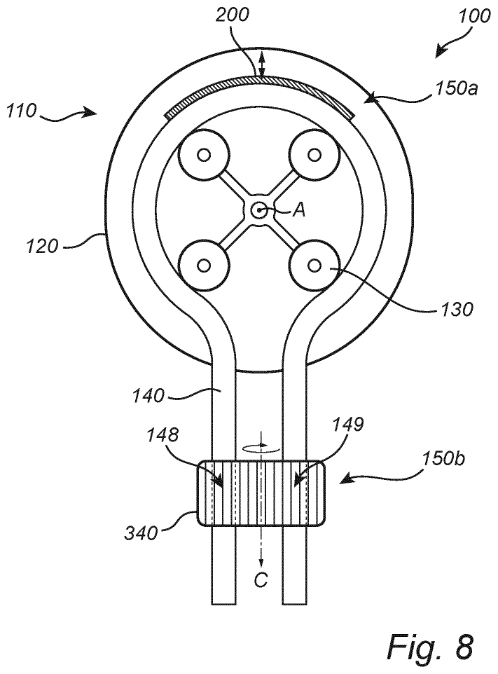

FIG. 8, schematically shows a drive assembly 100 according to an exemplifying embodiment of the present invention. It should be noted that the drive assembly 100 shown in FIG. 8 has several features in common with the drive assembly 100 shown in FIG. 1A-B and FIG. 2-5, and it is hereby referred to FIG. 1A-B and FIG. 4-5, and the associated texts for an increased understanding of some of the features and/or functions of the drive assembly 100.

In FIG. 8 the delivery control mechanism 150 comprises a control element 340 arranged to rotate around a third axis, C, parallel to the second axis, B. The control element 340 is arranged circumferentially around the third axis, C, and it at least partially encloses an outlet breathable section (not shown) arranged outside the pump housing 120. An inlet segment 148 of the flexible tube 140 arranged on an inlet side of the peristaltic pump assembly 110 comprises an inlet breathable section (not shown). The inlet breathable section may be the breathable section as illustrated in FIG. 5 and 6A-B and described in the associated texts. An outlet segment 149 of the flexible tube 140 arranged on an outlet side of the peristaltic pump assembly 110 comprises the outlet breathable section.

The flexible tube 140 comprises a breathable section 144 arranged outside the pump housing 120 and configured to be permeable to gas. The delivery control mechanism 150 comprises a rotating element 310a arranged circumferentially around a segment of the flexible tube 140 comprising the breathable section 144, and configured to rotate relative the flexible tube 140, around a second axis, B.

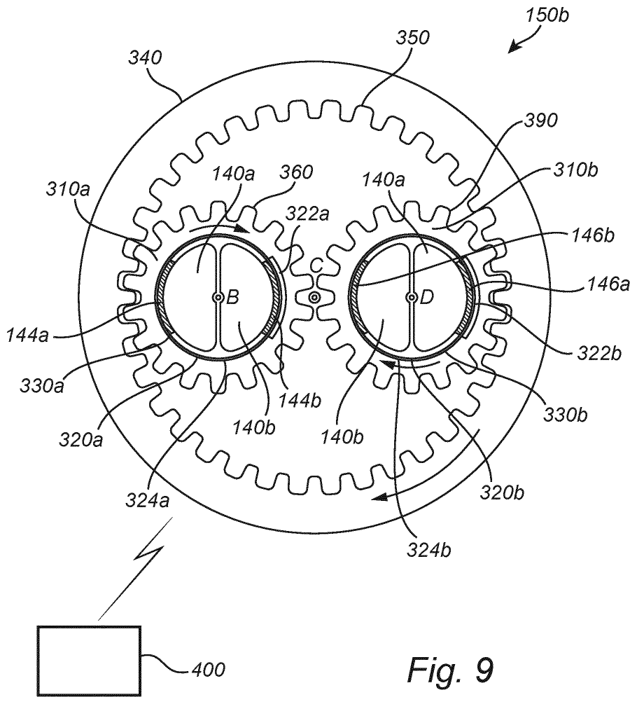

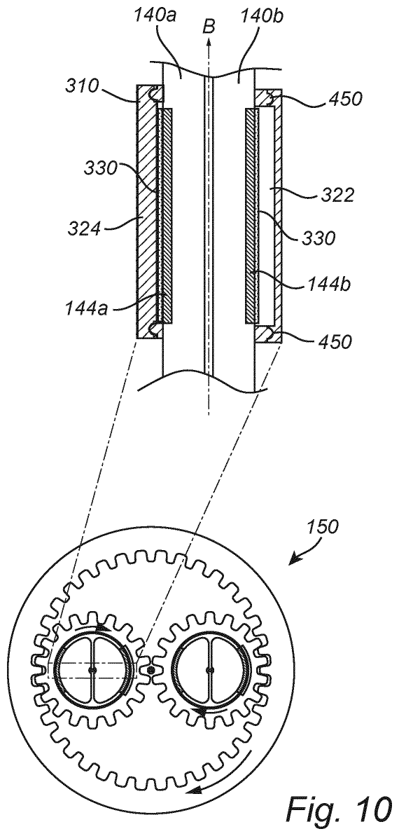

Furthermore, in FIG. 8, the delivery control mechanism 150 comprises a first delivery control mechanisms 150a and a second delivery control mechanism 150b. The first delivery control mechanism 150a comprises a lid element 200 arranged inside the pump housing 120. For further explanations of the first delivery mechanism 150a with the lid element 200 see FIG. 1-4 and associated texts. The second delivery control mechanism 150b comprises a control element 340 and a rotating element (not shown). For further explanations of the second delivery mechanism 150b with the rotating element, see FIG. 5-7. The second delivery mechanism 150b and the control element 340 will be further described in FIG. 9-10 and associated texts.

FIG. 9 schematically shows a cross-section of a delivery control mechanism 150b and two flexible tubes 140a, 140b according to an exemplifying embodiment of the present invention. It should be noted that the delivery control mechanism 150b shown in FIG. 9 has several features in common with delivery control mechanism 150 shown in FIG. 5-7, and it is hereby referred to FIG. 5-7, and the associated texts for an increased understanding of some of the features and/or functions of the delivery control mechanism 150b.

In FIG. 9, the drive assembly comprises two flexible tubes 140a, 140b. The flexible tubes 140a, 140b are attached to one another. It is to be understood that the flexible tubes 140a, 140b may constitute one flexible tube with two separate lumens. The flexible tubes 140a, 140b may be as illustrated and described in FIG. 8 and the associated text. The flexible tubes 140a, 140b comprises two outlet breathable sections 146a, 146b arranged outside the pump housing (not shown) and configured to be permeable to gas. An inlet segment (reference 148 in FIG. 8), of the flexible tubes 140a, 140b is arranged on an inlet side of the peristaltic pump assembly and comprises a first and second breathable sections 144a, 144b. An outlet segment, with reference 149 in FIG. 8, of the flexible tubes 140 a, 140 b is arranged on an outlet side of the peristaltic pump assembly (not shown) and comprises a first and second outlet breathable sections 146a, 146b. The inlet and outlet segments are segments of the same merged flexible tubes 140a, 140b arranged on different sides of the peristaltic pump assembly, i.e. an inlet side and an outlet side.

The delivery control mechanism 150b comprises a control element 340. The control element 340 is arranged to rotate around a third axis, C, parallel to the second axis, B, and the fourth axis, D. The delivery control mechanism 150b comprises a rotating element 310a and a second rotating element 310b arranged inside the control element 340. The rotating element 310a and the second rotating element 310b may have the same design, e.g. have the same parts, sizes, shapes and configuration. The rotating elements 310a, 310b may be identical.

The rotating element 310a is arranged circumferentially around the inlet segment. The inlet segment, represented as 148 in FIG. 8, comprises the first and second breathable sections 144a, 144b. The rotating element 310a comprises an inner surface 320a facing the flexible tubes 140a, 140b, wherein the inner surface 320a comprises a recess 322a.

The rotating element 310a is configured to rotate relative the two flexible tubes 140a, 140b, around the second axis, B, wherein the recess 322a is configured to be alignable with the first and second breathable section 144a, 144b when the rotating element 310a is rotated around the second axis, B.

The delivery control mechanism 150 comprises a sealing element 330a arranged circumferentially around the second axis, B, between the inner surface 320a and the flexible tube 140a, 140b, wherein the sealing element 330a completely covers the breathable section 144a.

The rotating element 310a is configured to rotate between a third position and a fourth position. In the third position, the recess 322a at least partially aligns with the first breathable section 144a and the second breathable section 144b is unaligned with the recess 322a. In the fourth position the recess 322a at least partially aligns with the second breathable section 144b and the first breathable section 144a is unaligned with the recess 322a.

The inner surface 320a comprises an abutting portion 324a arranged to abut the sealing element 330a and/or the flexible tubes 140 and cover the first breathable section 144a when the rotating element 310a is in the third position.

The second rotating element 310b is arranged circumferentially around the outlet segment comprising the first and second outlet breathable sections 146a, 146b. The rotating element 310b comprises an inner surface 320b facing the flexible tubes 140a, 140b, wherein the inner surface 320b comprises a recess 322b.

The rotating element 310b is configured to rotate relative the two flexible tubes 140a, 140b, around the fourth axis, D, wherein the recess 322b is configured to be alignable with the first and second outlet breathable section 146a, 146b when the rotating element 310b is rotated around the second axis, B.

The delivery control mechanism 150b comprises a sealing element 330b arranged circumferentially around the second axis, B, between the inner surface 320b and the flexible tube 140a, 140b, wherein the sealing element 330b completely covers the outlet breathable sections 146a, 146b.

The rotating element 310b is configured to rotate between a third position and a fourth position. In the third position, the recess 322b at least partially aligns with the first outlet breathable section 146a and the second outlet breathable section 146b is unaligned with the recess 322b. In the fourth position the recess 322b at least partially aligns with the second outlet breathable section 146b and the first outlet breathable section 146a is unaligned with the recess 322b.

The inner surface 320b comprises an abutting portion 324b arranged to abut the sealing element 330b and/or the flexible tubes 140 and cover the first outlet breathable section 146a when the rotating element 310b is in the third position.

The control element 340 comprises first cogs 350 on a surface facing the rotating elements 310a,310b. The rotating element 310a comprises second cogs 360 on a surface facing the first cogs 350. The second rotating element 310b comprises third cogs 390 on a surface facing the first cogs 350. The first cogs 350 are arranged to engage the second cogs 360 and the third cogs 390 when the control element 340 is rotated around the third axis, C, such that the flexible tubes 140a, 140b may be switched between the delivery state and the non-delivery state at the breathable section 144a, 144b and the outlet breathable section 146a, 146b simultaneously when the control element 340 is rotated.

The control element 340, the rotating element 310a and the second rotating element 310b may be configured to be synchronized/coordinated, e.g. by the design of the cogs, such that the flexible tube 140a may be switched to a non-delivery state by both the selective engagement at the first breathable section 144a and the first outlet breathable section 146a if the rotating elements 310a, 310b are in the third position and the recesses 322a, 322b are aligned with the breathable sections 144a, 146a. Accordingly, the second rotating element 310b may be configured to be synchronized/coordinated such that the flexible tube 140b may be switched to a non-delivery state by both the selective engagement at the second breathable section 144b and the second outlet breathable section 146b if the rotating elements 310a, 310b are in the fourth position and the recesses 322a, 322b are aligned with the breathable sections 144b, 146b.

The control element 340 may be connected/coupled to a motor (not shown) so that the control element 340 may be rotated by the motor and/or manually rotated by an operator. The delivery control mechanism 150 may comprise the motor, e.g. in connection with the control element 340.

In FIG. 9, the drive assembly further comprises a second control unit 400 configured to control the rotation of the control element 340 around the third axis, C. The control element 340 may also be operated manually.

FIG. 10 schematically shows a cross-section of a delivery control mechanism 150 according to an exemplifying embodiment of the present invention. It should be noted that the delivery control mechanism 150 shown in FIG. 9 has several features in common with delivery control mechanism 150 shown in FIG. 5-9, and it is hereby referred to FIG. 5-9, and the associated texts for an increased understanding of some of the features and/or functions of the delivery control mechanism 150.

FIG. 10 illustrates a zoomed cross section extending along the second axis, B of the rotating element 310 and the flexible tube 140. The delivery control mechanism 150 comprises a rotating element 310, a sealing element 330, a recess 322 and an abutting portion 324. The drive assembly comprises two flexible tubes 140a, 140b, wherein the two flexible tubes may constitute one flexible tube with two lumens. Each lumen/flexible tube may be connected to its own medicament container of the medicament delivery device. The rotating element 310 is arranged circumferentially around the flexible tubes 140a, 140b and the second axis, B. The sealing element 330 is arranged between the rotating element 310 and the flexible tubes 140a, 140b. The sealing element 330 is at least partially enclosed by the rotating element 310. The delivery control mechanism 150 may comprise an attaching part 450. The attaching part 450 is configured to fixedly attach the delivery control mechanism 150, e.g. the rotating element 310, to the flexible tube 140a, 140b. The attaching part 450 may comprise an adhesive. The attaching part 450 may be configured to mechanically grip/hold on to the flexible tube 140a, 140b, such that the delivery control mechanism 150 is better kept in place. The attaching part 450 may be part of the rotating element 310. The attaching part 450 may comprise an annular shape and be arranged circumferentially around the second axis, B. The rotating element 310 may further comprise at least one vent/opening (not shown) fluidly connected with the recess 322, and a space outside the recess 322, for exhaust of gas leaving the flexible tube 140b and/or for entry of gas into the recess 322.

Additionally, variations to the disclosed examples can be understood and effected by the skilled person in practicing the claimed invention, from a study of the drawings, the disclosure, and the appended claims. The mere fact that certain measures are recited in mutually different dependent claims does not indicate that a combination of these measures cannot be used to advantage.

A feature described in relation to one aspect may also be incorporated in other aspects, and the advantage of the feature is applicable to all aspects in which it is incorporated. Other objectives, features, and advantages of the present inventive concept will appear from the detailed disclosure, from the attached claims as well as from the drawings.

Generally, all terms used in the claims are to be interpreted according to their ordinary meaning in the technical field, unless explicitly defined otherwise herein. Further, the use of terms “first”, “second”, and “third”, and the like, herein do not denote any order, quantity, or importance, but rather are used to distinguish one element from another. All references to “a/an/the [element, device, component, means, step, etc.]” are to be interpreted openly as referring to at least one instance of said element, device, component, means, step, etc., unless explicitly stated otherwise.

Other aspects of the invention disclosed in the following clauses.
1. A drive assembly (100) for a medicament delivery device, the drive assembly comprising:
   a peristaltic pump assembly (110) comprising:
      a pump housing (120),
      a roller (130) at least partially arranged inside the pump housing and configured to rotate around a first axis, A,
   a flexible tube (140) extending between a tube inlet (141) and a tube outlet (142), wherein the tube inlet is configured to be attached to a medicament container of the medicament delivery device, wherein at least a portion of the flexible tube is arranged transverse to the first axis, A, between the pump housing and the roller
   a delivery control mechanism (150), configured to selectively switch the flexible tube between a delivery state and a non-delivery state by selectively engaging the flexible tube,
   wherein, in the delivery state, the rotation of the roller around the first axis, A, can move medicament contained within the medicament container and/or the flexible tube from the tube inlet to the tube outlet, by the rotation of the roller, and
   wherein, in the non-delivery state, medicament contained within the medicament container and/or the flexible tube cannot be moved from the tube inlet to the tube outlet by the rotation of the roller around the first axis, A.
2. The drive assembly according to clause 1, wherein the delivery control mechanism is configured to selectively switch the flexible tube between a plurality of delivery states and a non-delivery state by selectively engaging the flexible tube, wherein each of the plurality of delivery states may provide a different pump rate for the peristaltic pump assembly.
3. The drive assembly according to clause 1 or 2, wherein the delivery control mechanism comprises
   a lid element (200) arranged inside the pump housing and configured to be movable relative the roller and the flexible tube in a direction transverse to the first axis, A, between a first lid position and second lid position, wherein in the first lid position a portion of the flexible tube is compressed between the roller and the lid element, and wherein in the second lid position the lid element is disengaged from the flexible tube, such that the flexible tube is in the delivery state when the lid element is in the first lid position and the flexible tube is in the non-delivery state when the lid element is in the second lid position.
4. The drive assembly according to clause 3, wherein the delivery control mechanism comprises a motor (210) and/or a magnet unit (220), wherein the motor and the magnet unit are configured to move the lid element between the first and second lid position.
5. The drive assembly according to clause 4, wherein the magnet unit is arranged in the pump housing, and wherein the lid element comprises a magnetic material, such that the magnet unit is arranged to provide a force on the lid element in a direction from the first lid position to the second lid position and/or in a direction from the second lid position to the first lid position.
6. The drive assembly according to clause 4 or 5, wherein the delivery control mechanism comprises
   a screw element (230) configured to rotate around a fifth axis, E, parallel to the first axis, A,
   the motor (210), wherein the motor is configured to rotate the screw element around the fifth axis, E, and
   a coupling part (240) coupled to the screw element and the lid element,
   wherein the screw element is configured to move the coupling part when the screw element is rotated around the fifth axis, E, in a direction perpendicular to the fifth axis, and wherein the lid element is moved between the first and second lid position when the screw element rotates and the coupling part is moved.
7. The drive assembly according to clause 6, wherein the lid element comprises at least one protrusion (202), and the coupling part is coupled to the protrusion.
8. The drive assembly according to clause 6 or 7, wherein the screw element comprises a first set of threads (232) and the coupling part comprises a first set of grooves (242), and wherein the first set of threads are configured to engage the first set of grooves, and wherein the screw element is configured to move the coupling part in a direction perpendicular to the fifth axis, E, by rotating around the fifth axis, E.
9. The drive assembly according to any one of clauses 4 to 8, wherein the delivery control mechanism comprises a motor and a magnet unit, and wherein the motor is configured to move the lid element in a first direction, and wherein the magnet unit is configured to provide a force on the lid element in the first direction or in a second direction opposite the first direction.
10. The drive assembly according to any one of the preceding clauses, wherein the peristaltic pump assembly comprises a shaft (260) extending along the first axis, A, and configured to rotate around the first axis, A, and wherein the roller is arranged on the shaft and rotated around the first axis, A, when the shaft rotates.
11. The drive assembly according to clause 10, wherein the drive assembly further comprises a motor (270), wherein the shaft is coupled to the motor, and the motor is configured to rotate the shaft around the first axis, A.
12. The drive assembly according to any one of clauses 3 to 11, wherein the peristaltic pump assembly comprises at least two flexible tubes, and wherein the delivery control mechanism comprises at least two lid elements, and wherein each tube of the at least two flexible tubes can be compressed by a corresponding lid element of the at least two lid elements when the corresponding lid element is in the first lid position.
13. The drive assembly according to clause 12, wherein at least a portion of a first flexible tube is arranged transverse to the first axis, A, between a first lid element and the roller, and at least a portion of a second flexible tube is arranged transverse to the first axis, A, between a second lid element and the roller, wherein the first lid element is configured to be movable relative the roller and the first flexible tube in a direction transverse to the first axis, A, between the first lid position and the second lid position, and wherein the second lid element is configured to be movable relative the roller and second flexible tube in a direction transverse to the first axis, A, between the first lid position and the second lid position.
14. The drive assembly according to clause 13, wherein the first and second lid element may be moved between the first and second lid position individually.
15. The drive assembly according to any one of clauses 12 to 14, wherein the drive assembly further comprises a first control unit (280) configured to control the movement of the lid elements between the first and second lid position.
16. The drive assembly according to any one of the preceding clauses, wherein the flexible tube comprises a breathable section (144) arranged outside the pump housing and configured to be permeable to gas, and wherein the delivery control mechanism comprises
   a rotating element (310a) arranged circumferentially around the flexible tube comprising the breathable section, and configured to rotate relative the flexible tube, around a second axis, B, wherein the rotating element comprises an inner surface (320a) facing the flexible tube, and wherein the inner surface comprises a recess (322a), and
   a sealing element (330a) arranged circumferentially around the second axis, B, between the inner surface and the flexible tube, wherein the sealing element completely covers the breathable section,
   wherein the rotating element is configured to rotate between a first position and a second position, wherein in the first position the recess at least partially aligns with the breathable section, and wherein in the second position the breathable section is unaligned with the recess, such that the flexible tube is in the non-delivery state when the rotating element is in the first position and the flexible tube is in the delivery state when the rotating element is in the second position.
17. The drive assembly according to clause 16, wherein the sealing element is attached to the inner surface and covers the recess.
18. The drive assembly according to clause 16, wherein the sealing element is flexible and is attached to the flexible tube.
19. The drive assembly according to any one of clause 16 to 18, wherein the inner surface comprises an abutting portion (324a) arranged to abut the sealing element and/or the flexible tube and cover the breathable section when the rotating element is in the second position, such that a gas-tight seal is created with the sealing element for the breathable section.
20. The drive assembly according to any one of clauses 16 to 19, wherein the recess comprises a channel exiting the rotating element.
21. The drive assembly according to any one of clauses 16 to 20 wherein the drive assembly comprises two flexible tubes (140a, 140b), and
   wherein the flexible tubes (140a, 140b) are attached to one another, and wherein the flexible tubes extend between a respective tube inlet and a tube outlet, wherein each of the tube inlets is configured to be attached to a respective medicament container of the medicament delivery device, wherein at least a portion of each of the flexible tubes is arranged transverse to the first axis, A, between the pump housing and the roller, and
   wherein a first of the two flexible tubes comprises a first breathable section (144a) and a second of the two flexible tubes comprises a second breathable section (144b), and
   wherein the rotating element is arranged circumferentially around a segment of the two flexible tubes comprising the first and second breathable sections, and configured to rotate relative the two flexible tubes, around the second axis, B, wherein the recess of the inner surface is configured to be alignable with the first and second breathable section, and
   wherein the rotating element is configured to rotate between a third position and a fourth position,
   wherein in the third position, the recess at least partially aligns with the first breathable section and the second breathable section is unaligned with the recess, and
   wherein in the fourth position the recess at least partially aligns with the second breathable section and the first breathable section is unaligned with the recess,
   such that the first of the two flexible tubes is in the non-delivery state and the second of the two flexible tubes is in the delivery state when the rotating element is in the third position, and the second of the two flexible tubes is in the non-delivery state and the first of the two flexible tubes is in the delivery state when the rotating element is in the fourth position.
22. The drive assembly according to any one of clauses 16 to 21, wherein the delivery control mechanism comprises a control element (340) arranged to rotate around a third axis, C, parallel to the second axis, B, and
   wherein the control element comprises first cogs (350) on a surface facing the rotating element, and
   wherein the rotating element comprises second cogs (360) on a surface facing the first cogs, and wherein the first cogs are arranged to engage the second cogs when the control element is rotated around the third axis, C.
23. The drive assembly according to clause 22, wherein the control element is connected to a motor so that the control element may be rotated by the motor and/or manually rotated by an operator.
24. The drive assembly according to clause 22 or 23, wherein the flexible tube comprises an outlet breathable section (146) arranged outside the pump housing and configured to be permeable to gas, wherein an inlet segment (148) of the flexible tube arranged on an inlet side of the peristaltic pump assembly comprises the breathable section and an outlet segment (149) of the flexible tube arranged on an outlet side of the peristaltic pump assembly comprises the outlet breathable section, and
   wherein the delivery control mechanism further comprises a second rotating element (310b) arranged circumferentially around the outlet segment of flexible tube comprising the outlet breathable section, and configured to rotate relative the flexible tube, around a fourth axis, D, wherein the rotating element comprises a second inner surface (320b) facing the flexible tube, and wherein the inner surface comprises a second recess (322b), and
   a second sealing element (330b) arranged circumferentially around the fourth axis, D, between the inner surface and the flexible tube, wherein the second sealing element completely covers the outlet breathable section,
   wherein the second rotating element is configured to rotate between a first position and a second position, wherein in the first position the second recess at least partially aligns with the outlet breathable section, and wherein in the second position the outlet breathable section is unaligned with the second recess, such that the flexible tube is in the non-delivery state when the rotating element is in the first position and the flexible tube is in the delivery state when the rotating element is in the second position
   wherein the second rotating element further comprises third cogs (390) on a surface facing the first cogs,
   wherein the first cogs are arranged to engage the second cogs and the third cogs when the control element is rotated around the third axis, C, such that the flexible tube may switch to the delivery state or the non-delivery state by simultaneous selective engagement at the breathable section and at the outlet breathable section.
25. The drive assembly according to clause 23 or 24, wherein the drive assembly further comprises a second control unit (400) configured to control the rotation of the control element around the third axis, C.

1-15. (canceled)

16. A drive assembly for a medicament delivery device, the drive assembly comprising:
a peristaltic pump assembly comprising:
a pump housing; and
a roller at least partially arranged inside the pump housing and configured to rotate around a first axis;
a flexible tube extending between a tube inlet and a tube outlet, wherein the tube inlet is configured to be attached to a medicament container of the medicament delivery device, wherein at least a portion of the flexible tube is arranged transverse to the first axis, A, between the pump housing and the roller; and
a delivery control mechanism configured to selectively switch the flexible tube between a delivery state and a non-delivery state by selectively engaging the flexible tube, wherein, in the delivery state, the rotation of the roller around the first axis can move medicament contained within the medicament container and/or the flexible tube from the tube inlet to the tube outlet, by the rotation of the roller, and
wherein, in the non-delivery state, medicament contained within the medicament container and/or the flexible tube cannot be moved from the tube inlet to the tube outlet by the rotation of the roller around the first axis.

17. The drive assembly according to claim 16, wherein the delivery control mechanism comprises:
a lid element arranged inside the pump housing and configured to be movable relative the roller and the flexible tube in a direction transverse to the first axis, between a first lid position and second lid position, wherein in the first lid position a portion of the flexible tube is compressed between the roller and the lid element, and wherein in the second lid position the lid element is disengaged from the flexible tube, such that the flexible tube is in the delivery state when the lid element is in the first lid position and the flexible tube is in the non-delivery state when the lid element is in the second lid position.

18. The drive assembly according to claim 17, wherein the delivery control mechanism comprises a motor and/or a magnet unit, wherein the motor and the magnet unit are configured to move the lid element between the first and second lid position.

19. The drive assembly according to claim 18, wherein the magnet unit is arranged in the pump housing, and wherein the lid element comprises a magnetic material, such that the magnet unit is arranged to provide a force on the lid element in a direction from the first lid position to the second lid position and/or in a direction from the second lid position to the first lid position.

20. The drive assembly according to claim 18, wherein the delivery control mechanism comprises:
a screw element configured to rotate around a fifth axis parallel to the first axis;
the motor, wherein the motor is configured to rotate the screw element around the fifth axis; and
a coupling part coupled to the screw element and the lid element, wherein the screw element is configured to move the coupling part when the screw element is rotated around the fifth axis, in a direction perpendicular to the fifth axis, and wherein the lid element is moved between the first and second lid position when the screw element rotates and the coupling part is moved.

21. The drive assembly according to claim 20, wherein the lid element comprises at least one protrusion, and the coupling part is coupled to the protrusion.

22. The drive assembly according to claim 20, wherein the screw element comprises a first set of threads and the coupling part comprises a first set of grooves, and wherein the first set of threads are configured to engage the first set of grooves, and wherein the screw element is configured to move the coupling part in a direction perpendicular to the fifth axis, by rotating around the fifth axis.

23. The drive assembly according to claim 18, wherein the delivery control mechanism comprises a motor and a magnet unit, and wherein the motor is configured to move the lid element in a first direction, and wherein the magnet unit is configured to provide a force on the lid element in the first direction or in a second direction opposite the first direction.

24. The drive assembly according to claim 16, wherein the delivery control mechanism is configured to selectively switch the flexible tube between a plurality of delivery states and a non-delivery state by selectively engaging the flexible tube, wherein each of the plurality of delivery states may provide a different pump rate for the peristaltic pump assembly.

25. The drive assembly according to claim 16, wherein the peristaltic pump assembly comprises a shaft extending along the first axis, and configured to rotate around the first axis, and wherein the roller is arranged on the shaft and rotated around the first axis, when the shaft rotates.

26. The drive assembly according to claim 25, wherein the drive assembly further comprises a motor, wherein the shaft is coupled to the motor, and the motor is configured to rotate the shaft around the first axis.

27. The drive assembly according to claim 18, wherein the peristaltic pump assembly comprises at least two flexible tubes, and wherein the delivery control mechanism comprises at least two lid elements, and wherein each tube of the at least two flexible tubes can be compressed by a corresponding lid element of the at least two lid elements when the corresponding lid element is in the first lid position.

28. The drive assembly according to claim 27, wherein at least a portion of a first flexible tube is arranged transverse to the first axis between a first lid element and the roller, and at least a portion of a second flexible tube is arranged transverse to the first axis, between a second lid element and the roller, wherein the first lid element is configured to be movable relative the roller and the first flexible tube in a direction transverse to the first axis, between the first lid position and the second lid position, and wherein the second lid element is configured to be movable relative the roller and second flexible tube in a direction transverse to the first axis, between the first lid position and the second lid position.

29. The drive assembly according to claim 15, wherein the first and second lid element may be moved between the first and second lid position individually.

30. The drive assembly according to claim 27, wherein the drive assembly further comprises a first control unit configured to control the movement of the lid elements between the first and second lid position.
